(19) United States
(12) Patent Application Publication
(10) Pub. No.: US 2026/0257067 A1
(43) Pub. Date: Sep. 3, 2026

(54) Systems and Methods for Vagus Nerve Stimulation with Miniaturized Wirelessly Powered Stimulator

(71) Applicant: The Regents of the University of California, Oakland, CA (US)

(72) Inventors: Aydin Babakhani, Los Angeles, CA (US); Iman Habibagahi, Los Angeles, CA (US)

(73) Assignee: The Regents of the University of California, Oakland, CA (US)

(21) Appl. No.: 18/718,405

(22) PCT Filed: Dec. 12, 2022
(86) PCT No.: PCT/US2022/081388

§ 371 Date: Jun. 10, 2024

Related U.S. Application Data
(60) Provisional application No. 63/265,215, filed on Dec. 10, 2021.

(51) Int. Cl. A61N 1/36; A61N 1/372; A61N 1/378

(52) U.S. Cl. CPC A61N 1/36114; A61N 1/36139; A61N 1/37235; A61N 1/3787

(57) ABSTRACT

Systems and methods for vagus nerve stimulation using wirelessly powered stimulators in a closed-loop monitoring system in accordance with embodiments of the invention are disclosed. One embodiment includes a method for vagus nerve stimulation, the method includes implanting at least one wirelessly powered stimulator proximate to a vagus nerve of a subject, receiving a control input describing stimulation data from a controller device, providing a radio frequency (RF) signal from the wearable device to the stimulator based on the control input, receiving and recovering power from the RF signal using the stimulator, outputting a stimulation that releases the energy stored in the energy storage capacitor on a plurality of electrodes in an output pulse having characteristics based on the received RF signal, monitoring at least one vital sign of the subject while outputting the stimulation, and adjusting the outputting the stimulation based on the monitored at least one vital sign.

### CROSS-REFERENCE TO RELATED APPLICATIONS

The current application claims the benefit of and priority under 35 U.S.C. § 119(e) to U.S. Provisional Patent Application No. 63/265,215 entitled “Systems and Methods for Vagus Nerve Stimulation with Miniaturized Wirelessly Powered Stimulator” filed Dec. 10, 2021, the disclosures of which is hereby incorporated by reference in its entirety for all purposes.

### FIELD OF THE INVENTION

The present invention relates generally to vagus nerve stimulation (VNS) and more specifically to closed-loop monitoring systems for VNS using wirelessly powered stimulators.

### BACKGROUND OF THE INVENTION

The vagus nerve is an important cranial nerve connecting the brainstem to the body. It allows the brain to monitor and interface with several of the body's most critical functions. Some key functions of the vagus nerve include providing taste sensation behind one's tongue, providing movement functions for neck muscles responsible for swallowing and speech, as well as controlling a person's digestive tract, respiration, and heart rate.

Wireless power transmission refers to the transmission of electrical energy without wires as a physical connection. There are two main categories of wireless power techniques: near field and far field. An example of near field wireless power technique is inductive coupling, where power is transferred over a short distance through coils of wire or electric fields using capacitive coupling between metal electrodes.

Implantable pulse generators (IPGs) have solved various critical clinical problems and improved the quality of human life. Their applications can include chronic pain relief, motor function recovery for spinal cord injuries, the treatment of gastroesophageal reflux disease, cardiac pacemaking, and curing stress urinary incontinence, among various other applications. Conventional IPGs are bulky with the battery taking up most of the unit, and the necessary leads are prone to cause various complications.

Closed-loop monitoring systems use variables being measured by the system as controlled variables to regulate the system. It can provide accurate monitoring and regulation in real-time to optimize system performance.

### SUMMARY OF THE DISCLOSURE

Systems and methods for vagus nerve stimulation (VNS) of a subject (e.g., a patient) using wirelessly powered stimulators in a closed-loop monitoring system in accordance with embodiments of the invention are disclosed. One embodiment includes a method for vagus nerve stimulation using at least one wirelessly powered stimulator, where the method includes implanting at least one wirelessly powered stimulator proximate to a vagus nerve of a subject, each wirelessly powered stimulator includes: an implantable pulse generator (IPG), including: an Rx antenna that receives a radio frequency (RF) signal from an external Tx antenna, a rectifier, an energy storage capacitor C_{STORAGE}, where the RF signal coupled to the Rx antenna is rectified by the rectifier to generate VDD and charges the C_{STORAGE}, a demodulator, an output voltage regulator that generates a stable voltage to activate the demodulator; and where the demodulator outputs a stimulation that releases the energy stored in the C_{STORAGE} on a cuff electrode based on detecting amplitude modulation in the received RF signal, and a Tx antenna that generates the RF signal that wirelessly powers the IPG and that controls timing of output stimulations of the IPG, where amplitude modulation is applied to the RF signal to control the timing of the output stimulations. The method further includes receiving, at a wearable device, a control input describing stimulation data from a controller device, providing a radio frequency (RF) signal from the wearable device to the stimulator based on the control input, receiving and recovering power from the RF signal using the stimulator, outputting a stimulation that releases the energy stored in the energy storage capacitor on a plurality of electrodes in an output pulse having characteristics based on the received RF signal, monitoring, at the wearable device, at least one vital sign of the subject while outputting the stimulation, and adjusting the outputting the stimulation based on the monitored at least one vital sign.

In a further embodiment, the IPG further includes several reverse bias diodes that release energy from the C_{STORAGE} when the energy stored reaches an upper level threshold.

In a further embodiment again, the Rx antenna is at least one antenna selected from the group consisting of an inductor coil, a resonant coil, a dipole antenna, a monopole antenna, a patch antenna, a bow-tie antenna, a phased-array antenna, and a wire.

In still a further embodiment, the C_{STORAGE} is off-chip.

In a further embodiment still, the C_{STORAGE} is on-chip.

In a further embodiment again, the Rx antenna is off-chip.

In a further embodiment yet again, the Rx antenna is on-chip.

In yet a further embodiment, amplitude modulation includes detecting at least a threshold percentage reduction in power of the RF signal from the Tx antenna.

In still a further embodiment again, the IPG further includes a DC-block capacitor, C_{FILTER}, that delivers the output stimulations for charge-neutralization.

In still a further embodiment again still, the IPG further includes a discharge resistor, R_{DIS}, that nulls the accumulated charge on the C_{FILTER}.

In still a further embodiment yet again, the IPG is used for at least one application selected from the group consisting of neural stimulation, heart pacing, defibrillation, bladder stimulation and deep brain stimulation.

In yet still a further embodiment again, the output voltage regulator limits an amplitude of output stimulations within a specific range, where the output voltage regulator enables the demodulator when a supply voltage exceeds a lower tier, and where when the supply voltage exceeds a higher tier, enables a discharge path to rapidly discharge excess incident charge.

In still a further embodiment again, the amplitude modulation is applied to the RF signal to control at least one of a repetition rate and a duration of the output stimulation in an analog manner.

In still a further embodiment again, the demodulator replicates a timing of the amplitude modulation applied to the RF signal.

In still a further embodiment again, the demodulator includes three source follower replicas with a high end V_{H}, low end V_{L}, and transient envelop V_{ENV} of the RF signal and the V_{ENV} detection branch uses a small capacitor Cₛₘ and V_{H} and V_{L} are extracted on large capacitors with and without the AC input respectively.

In still a further embodiment again, an average of V_{H} and V_{L}, V_{M}, is obtained using a resistive divider and compared with V_{ENV} to reconstruct the timing of the amplitude modulation.

In still a further embodiment again, a recovered timing signal is sharpened by a buffer.

In still another embodiment again, the adjusting the outputting the stimulation based on the monitored at least vital sign includes: transmitting the monitoring results to from the wearable device to the controller device, adjusting, at the controller device, the control input based on the monitoring results, adjusting, at the wearable device, the RF signal based on the adjusted control input, and providing the adjusted RF signal to the stimulator.

In a still further embodiment again, the method includes outputting a stimulation that releases the energy stored in the energy storage capacitor on the plurality of electrodes in an output pulse having characteristics based on the received adjusted RF signal.

In yet another additional embodiment, the adjusting, at the controller device, the control input based on the monitoring results includes: determining a target heart rate, and determining a level of stimulation to maintain the heart rate of the subject at the target heart rate.

In a yet further additional embodiment, the adjusting of input is performed dynamically based on the monitoring results.

In yet another embodiment again, the dynamic adjustment of input is performed using a data-driven model.

In a yet further embodiment again, the at least one vital sign of the subject monitored by the wearable device is the subject's heart rate.

In another additional embodiment again, the at least one wirelessly powered stimulator includes a plurality of wireless powered stimulators and the vagus nerve stimulation is performed in more than one location by the plurality of wireless powered stimulations.

In a further additional embodiment again, the wirelessly powered stimulator is located within 5 cm of the wearable device.

In still yet another additional embodiment, the control device comprises an electrocardiogram (ECG) machine.

In still another embodiment again, the monitoring results comprises an ECG signal of the subject.

In a still further additional embodiment, the monitoring results are provided to a coronary care unit (CCU) through the wearable device.

In yet another additional embodiment, the wearable device is rechargeable.

In a yet further additional embodiment, the stimulation can be stopped by a fail-safe mechanism when the monitored vital sign indicates extreme heart rate deviations.

In another additional embodiment again, the characteristics of the output pulse determined by the RF signal are voltage, frequency, and pulse width.

One embodiment includes a system for vagus nerve stimulation, including a wearable device configured to receive a control input describing stimulation data, provide a radio frequency (RF) signal from the wearable device to the stimulator based on the control input, and monitor at least one vital sign of a subject implanted with at least one wirelessly powered stimulator while outputting the stimulation. The system further includes at least one wirelessly powered stimulator, each wirelessly powered stimulator including an implantable pulse generator including an Rx antenna, a rectifier, an energy storage capacitor, a demodulator, and an output voltage regulator, and each implantable pulse generator is configured to receive and recover power from the RF signal, and output a stimulation that releases the energy stored in the energy storage capacitor on a plurality of electrodes in an output pulse having characteristics based on the received RF signal. The system further includes a controller device configured to provide the control input describing stimulation data, and adjust and output the stimulation based on the monitored at least one vital sign.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A illustrates an overview of the system architecture of a vagus nerve stimulation system with closed-loop monitoring in accordance with an embodiment of the invention.

FIG. 1B illustrates an in vivo experiment in which an IPG is fully implanted and used to stimulate the pig's vagus nerve in accordance with an embodiment of the invention.

FIG. 2A illustrates a circuitry overview, with the circuit architecture of an IPG in accordance with an embodiment of the invention.

FIG. 2B illustrates a schematic of the Tx coil in accordance with an embodiment of the invention.

FIG. 3 illustrates a circuit schematic of a demodulator in accordance with an embodiment of the invention.

FIGS. 4A-C illustrates a circuit schematic of an output voltage regulator in accordance with an embodiment of the invention. In particular, FIGS. 4A and 4B illustrates setting the high and low bars of the output amplitude, respectively, and FIG. 4C generates the voltage reference in accordance with an embodiment of the invention.

FIG. 5 illustrates an overall current consumption of the IC and that of the individual blocks in accordance with an embodiment of the invention.

FIG. 6 illustrates a circuit model of an energy-harvesting frontend resonator in accordance with an embodiment of the invention.

FIG. 7A illustrates a 3D model of an implemented Rx coil in accordance with an embodiment of the invention.

FIG. 7B illustrates a model of an as-fabricated PCB incorporating an Rx coil in accordance with an embodiment of the invention.

FIG. 8A illustrates a simplified model of an energy-harvesting frontend resonator in accordance with an embodiment of the invention.

FIG. 8B illustrates a circuit schematic of a Dickson rectifier in accordance with an embodiment of the invention.

FIG. 9A illustrates a 3-dB bandwidth and FIG. 9B illustrates normalized Q_{η} for different rectifier designs in accordance with an embodiment of the invention.

FIG. 10 illustrates a simulated dependence of R_{REC} and C_{REC} On I_{LOAD} in accordance with an embodiment of the invention.

FIG. 11 illustrates a resonant frequency drift in muscle medium in accordance with an embodiment of the invention.

FIG. 12 illustrates a co-design procedure for the Rx coil and the rectifier, which ensures optimal performance at a specific Med Radio band in accordance with an embodiment of the invention.

FIG. 13 illustrates a microscopic image of a fabricated IC in accordance with an embodiment of the invention.

FIG. 14 illustrates a picture of an as-fabricated IPG assembly in comparison with a U.S. quarter in accordance with an embodiment of the invention.

FIG. 15A illustrates a picture of a Tx coil in accordance with an embodiment of the invention.

FIG. 15B illustrates the Tx coil's S11 according to measurement in accordance with an embodiment of the invention.

FIG. 16 illustrates an output voltage waveform of an IPG in response to a 100 μs notch in accordance with an embodiment of the invention.

FIG. 17A illustrates voltage (a, c) and the resulting current (b, d) waveforms for a 96.7 μs pulse and a 197.6 μs pulse, respectively. (e) Three cycles of 96.7 μs pulses at 10 Hz rate in accordance with an embodiment of the invention.

FIG. 17B illustrates output waveforms of an IPG with the LED loading the output in accordance with an embodiment of the invention.

FIG. 18 illustrates a process for vagus nerve stimulation with closed-loop monitoring in accordance with an embodiment of the invention.

FIG. 19A illustrates a model of animal experiment setup. The inset shows the implantation of the IPG in accordance with an embodiment of the invention. FIG. 19B illustrates a closer view of the implantation site where the skin will be sutured covering the device in accordance with an embodiment of the invention.

FIG. 20 illustrates the change in heart rate and periodicity of LVP in response to stimulation at various frequency with constant pulse width of 0.1 ms in accordance with an embodiment of the invention.

FIG. 21 illustrates simulated 10-g average SAR when the Tx coil is placed at a distance of 3 cm from the Rx coil in ANSYS in accordance with an embodiment of the invention.

FIG. 22 illustrates a table providing a comparison of recently published battery-less IPGs.

### DETAILED DESCRIPTION OF THE DRAWINGS

Vagus nerve stimulation (VNS) finds many applications in the treatment of epilepsy, refractory depression, and anxiety. In recent years, there have been new interests in VNS in treatments for visceral disorders and cardiac pathologies. VNS in bioelectric therapies has been shown to be capable of increasing parasympathetic tone, restoring reflexes that alleviate excessive adrenergic inputs to the heart, limiting cardiomyocyte apoptosis and inflammation, and altering substrate utilization within the heart muscle. VNS, when delivered to the cervical vagosympathetic trunk, activates both ascending (afferent) and descending (parasympathetic efferent) projections. The cardiac nervous system works in a “push push-back” fashion. Functional cardiac responses to afferent activation are engaged at lower stimulation, which leads to the withdrawal of centrally-derived parasympathetic tone with the potential to modify sympathetic activity. As stimulation increases, parasympathetic efferents are engaged with expected decreases in regional cardiac function.

When ascending and descending projections within the cervical vagus are activated in a “balanced” fashion, multiple levels of the cardiac neuraxis can be engaged with little or no change in basal cardiac function. This balance point is described as the neural fulcrum. VNS delivered at the neural fulcrum is able to place restraints on aberrant reflex processing within the peripheral neural networks of the intrinsic cardiac nervous system, render myocytes stress-resistant, and exert anti-adrenergic effects on the heart itself. Thus, to maintain the stimulation at the neural fulcrum, it is important to have closed-loop monitoring during VNS.

Systems and methods described herein attempt to achieve such a solution by presenting a closed-loop VNS system that is capable of monitoring the level of stimulation of a subject (e.g., a patient) and adjust as necessary in order to maintain stimulation at the neural fulcrum. In many embodiments, the stimulation is controlled dynamically based on the resulting heart rate and the ECG waveforms developed post-stimulation. This can be achieved through a sensing-stimulation closed-loop approach where the stimulation is governed and controlled to be in a well-defined window by sensing its effect and using an algorithm to control the stimulation parameters such as the frequency, pulse width, duty cycle, and amplitude of the stimulation signal to remain in the therapeutic target zone.

### VNS System Architecture

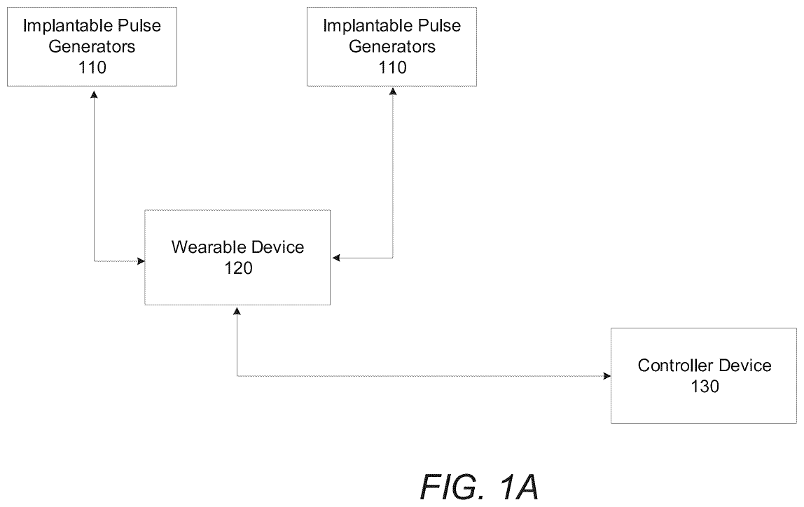

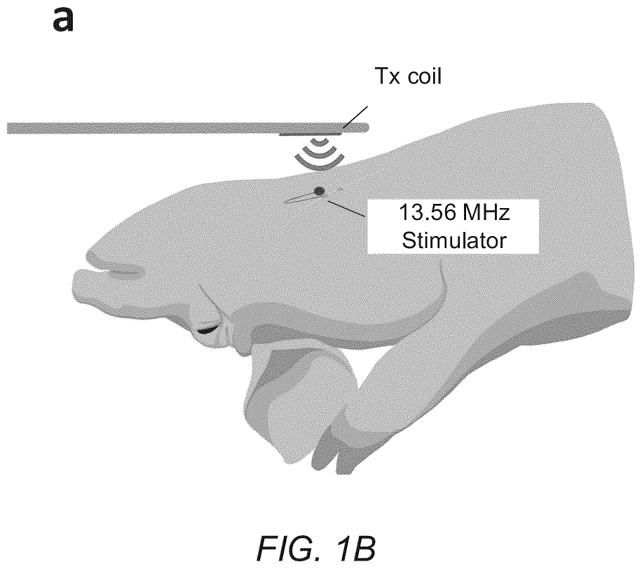

Turning now to the drawings, systems and methods for vagus nerve stimulation with closed-loop monitoring in accordance with various embodiments of the invention are illustrated. FIG. 1A illustrates a system architecture of a vagus nerve stimulation (VNS) system with closed-loop monitoring in accordance with embodiments of the invention. Many embodiments provide for achieving battery-less and leadless implantable pulse generators (IPGs) 110 that can be directly implanted in the specific anatomical region. In numerous embodiments, the VNS system includes a wearable device 120 that receives input from a controller device 130 that describes the desired stimulation intensity and transmits to the IPG 110. In many embodiments, IPGs 110 can sense stimulation effects on a subject while retaining a small form factor such that it may be integrated into the closed-loop VNS system. The wearable device 120 may monitor stimulation and receives feedback including sensed stimulation effects from the IPG 110. In some embodiments, the wearable device 120 transmits the monitoring results to the controller device 130. In some embodiments, the controller device 130 is used to adjust parameters of the stimulation to be delivered next based on the monitoring results. In several embodiments, the wearable device includes a coronary care unit (CCU).

In many embodiments, the wearable device includes a flexible wearable antenna that includes a Tx antenna that can operate at, for example, 13.56 MHz. The flexible antenna may be connected to an oscillator and microcontroller, which may be powered by a portable power source, such as a 3.2V battery. In some embodiments, the antenna is directly controlled by the CCU. The CCU may be programmed via wireless connection to the controller device, which may be a cellphone/laptop (e.g., Bluetooth, Wi-Fi, etc.) in real-time to control the stimulations' frequency, pulse width, and voltage. The power of the Tx antenna may be switched, for example from 100 mW to 1 W, in case more power is needed to power the implants.

The wearable device may have a battery that can be recharged, for example, using a 5V USB cable or inductive coupling. In many embodiments, 100 mW of power is sufficient to perform VNS where the wearable device and the IPG are 5 cm apart. By using a rechargeable battery with a capacity of 1200 mAh and a voltage of 3.2 V and assuming a reasonable transmitter efficiency of 70% (DC to RF), the entire VNS system can operate for 270 hours (11 days) continuously. In many embodiments, VNS may be performed on multiple sites on a test subject. Multisite VNS systems may be powered by a single 100 mW source powering the Tx antenna to operate at 13.56 MHz. This design satisfies the limits mandated in Federal Communications Commission sections 15.247 and 15.249 and will ensure that the transmitted power is within the safety limits set by IEEE. In many embodiments, a significant change in heart rate can be observed when the frequency of stimulation exceeds 2 Hz.

In several embodiments, electrocardiogram (ECG) signals are measured to calculate for the subject's heart rate (HR), another parameter of control for the VNS studies. ECG may be recorded non-invasively, making the wearable device extremely suitable for the task. A wearable sensor provides a much larger degree of freedom as it can be operated using a portable source of power, such as batteries, which can be replaced easily. In many embodiments, the wearable device 120 includes an ECG sensor that can sense an ECG signal from the subject during stimulation. The ECG signal may be digitized and transmitted from the wearable device 120 to the controller device 130. The CCU may process the data in real-time to extract the heart rate from the ECG signal to provide as input for closed-loop control for VNS. A new stimulation intensity may be generated based on the extracted HR, and VNS may proceed in the closed-loop control supplied by the CCU. Using the time difference between the peaks of R waves in the Q-wave, R-wave and S-wave (QRS) complex of the ECG signal, the HR of the subject can be accurately measured.

ECG signals from the wearable sensor may vary in strength and have varying signal characteristics depending on the location of the sensing node. In many embodiments, the ECG signals are filtered from the wearable sensor site and gain boosted such that useful signals to be used for HR calculation can be picked up. The received ECG signal may be filtered and transferred to PC/phone for additional monitoring and adjusting.

The CCU may take the monitored ECG signals and derive HR during VNS as inputs to provide stimulation parameters including amplitude, duration, duty cycle, and frequency to maintain outputting stimulation at neural fulcrum or null change in HR. Individual responses to VNS and changes in HR may be recorded to calibrate a target HR for a subject, and then the CCU adaptively controls the stimulation parameters to produce null HR change in stimulation. In many embodiments, fail-safe mechanisms to stop stimulation can stop stimulation until HR settles to a baseline in the event of extreme HR deviations.

Most stimulation devices function in either current or voltage modes. The current-controlled stimulation (CCS) provides precise current control irrelevant of the load impedance. However, because the stimulator needs to comply with the worst-case electrode/tissue impedance condition, the CCS renders the worse energy efficiency in most clinical settings. The voltage-controlled stimulation (VCS) regulates the stimulus in the voltage domain and renders an excellent energy efficiency. Due to this reason, most existing commercially available IPGs are based on VCS. A physician identifying the appropriate range of stimulus strength in advance and over time can reduce or eliminate the chance of overstimulation.

Wireless power transfer can be a power source in place of a battery to powers implantable medical devices (IMDs). Magnetic fields can penetrate tissues similar to how it travels in air. This makes it possible for wireless power transfer (WPT) to be achieved through resonant inductive coupling. Many current VNS systems rely on bulky batteries or an external wired power supply. WPT, in its current form factor, can be limited in range and data rate. Aside from far/mid-field coupling and ultrasonic transmission, the near-field inductive coupling is an attractive developing technology. The medical device radiocommunications (MedRadio) service, e.g., 401-406, 413-419, 426-432, 438-444, and 451-457 MHz, assigned by the federal communications commission has been used for the telemetry of IMDs. Unlike hundreds-MHz prior art that adopts on-chip coils, many embodiments of the IPG implement a miniaturized Rx coil on a PCB to minimize the cost. Also, in many embodiments of the IPG, a discrete energy storage capacitor is assembled with the integrated circuitry.

Accordingly, many embodiments provide a concise circuitry to realize an energy-efficient voltage-controlled IPG with a quiescent (while not stimulating) current consumption of 950 nA. In several embodiments, inductive coupling at a MedRadio band can achieve the wireless power link, where notches may be intentionally applied to precisely control the width and rate of the output pulses in an analog manner. In many embodiments, the energy-harvesting frontend circuitry takes account of the potential impacts of biological tissues. In many embodiments, the finalized assembly weighs 483 mg (81 mg without the cuff electrodes) with the diameter of the Rx coil being 13 mm. The potential use of an IPG in accordance with an embodiment of the invention was verified in an in vivo study in which the IPG was implanted at the neck of an anesthetized pig under the skin, as illustrated in FIG. 1B in accordance with an embodiment of the invention. In many embodiments, hemodynamic response in heart rate was observed during stimulation experiments.

Described are circuit implementations of IPGs in with a focus on the design tradeoffs in the energy-harvesting frontend circuitry in accordance with several embodiments of the invention. Furthermore, a discussion of the benchtop measurement and in vivo experiment results are provided.

### Circuit Implementation

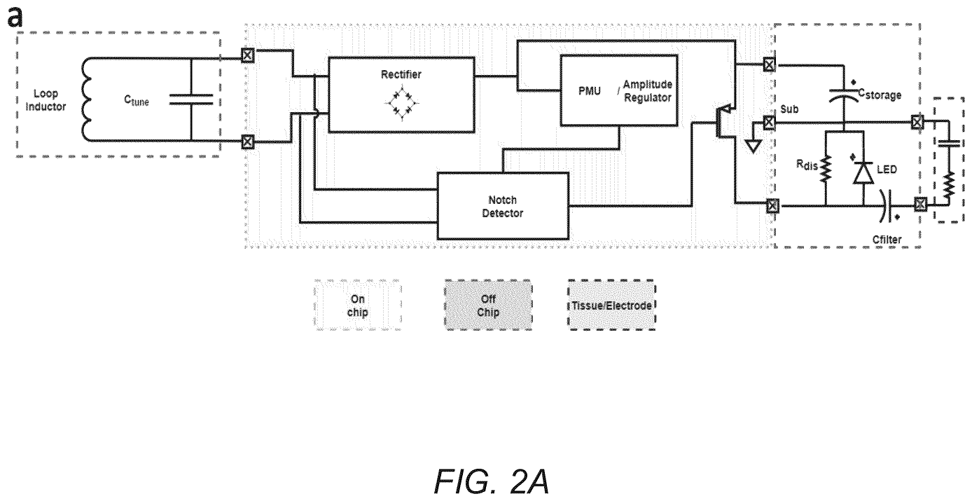

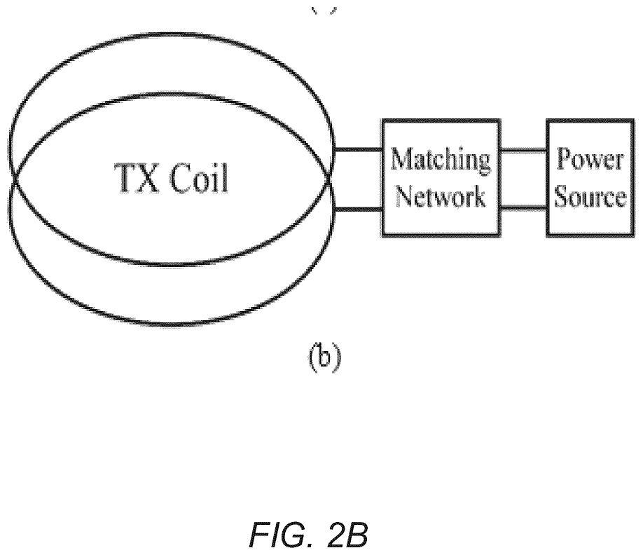

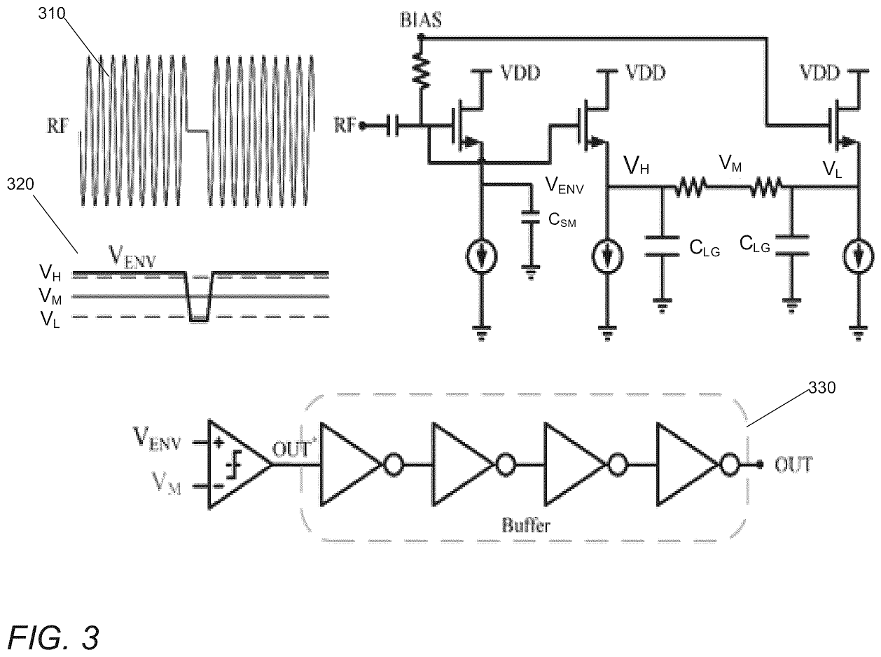

A system architecture of an IPG in accordance with an embodiment of the invention is shown in FIG. 2A. In many embodiments, the magnetic field coupled to the Rx coil can be rectified to generate VDD and charge an energy storage capacitor, C_{STORAGE}. In several embodiments, notches (e.g., RF power is reduced to a percentage of the RF power during harvest) can be intentionally applied in the Tx signal which precisely controls the timing of the output stimulations as their repetitions. The notch-based modulation scheme can eliminate any complex telemetry and minimizes the power consumption. In many embodiments, as the notches only constitute a negligible portion of the Tx power, they do not degrade the efficiency of the power transfer link. In many embodiments, a VCS scheme may be adopted for better energy-efficiency, in which VDD node can be directly applied to the electrode/tissue with a controllable pulse width. In many embodiments, in replacement of a low-dropout (LDO), a simplified output voltage regulator may be used to limit the amplitude of the output stimulations within a specific range, which may further reduce the static power consumption. In many embodiments, the regulator may enable the notch-demodulation block only when the supply voltage exceeds the lower tier. When the supply voltage exceeds the higher tier, a discharge path may be enabled to rapidly discharge the excess incident charge. The stimulations can be delivered through a DC-block capacitor, C_{FILTER}, for charge-neutralization. In several embodiments, a discharge resistor, R_{DIS}, nulls the accumulated charge on C_{FILTER}. A light-emitting diode (LED) can be optionally included at the output to visually identify that stimulation is occurring. Although FIG. 2A illustrates a particular circuit architecture of an IPG, any of a variety of circuit architectures may be utilized as appropriate to the requirements of specific applications in accordance with embodiments of the invention.

In many embodiments, there are 5 discrete components used on the stimulator PCB. The rectifier resonance frequency can be tuned using a tuning capacitor where C_{TUNE} equals 47 PF. The power is continuously harvested on a discrete storage capacitor where C_{STORAGE} equals 22 mF. To ensure that the charge is balanced, a series filtering capacitor with C_{FILTER} being 10 mF and parallel discharge resistor R_{DIS}=47 kΩ are assembled at the output.

In many embodiments, an IPG can be wirelessly powered and controlled by a custom Tx coil fabricated using a 1.6 mm FR4 substrate with 6 turns on each side. The Tx coil has a diameter of approximately 45 mm in accordance with an embodiment of the invention. In certain embodiments, the wideband Tx coil sweeps at different frequencies to find the resonant frequency of the Rx energy-harvesting frontend to achieve the minimum power to activate the output. In an embodiment of the invention, the resonant frequency for impedance matching was verified at approximately 13.56 MHz. To ensure maximum power delivery from the signal generator, the transmitter coil can be matched to 50 ohms. S11 measured using the VNS (PNA-L network analyzer) Model N5230C shows better than −38.4 dB matching and therefore a terminal efficiency higher than 99.99%. Although FIG. 2B illustrates a particular schematic of a Tx coil, any of a variety of architectures may be utilized as appropriate to the requirements of specific applications in accordance with embodiments of the invention.

In selected embodiments, the inductor on the receiver side is resonated with a high-quality factor (Q>200) 47 pF capacitor for maximum current delivery. Unlike the transmitter coil, the inductance cannot be directly measured due to the high parasitism of probes and the relatively small size of receiver coils. In an embodiment of the invention, the simulated quality factor for the Rx coil (Qr) is 65.2. The link efficiency is a function of mutual coupling (k) according to equation:

$$η \approx \frac{{K}^{2} {Q}_{t} Qr}{{( 1 + \sqrt{1 + {K}^{2} {Q}_{t} Qr} )}^{2}} \qquad ( 1 )$$

To maximize link efficiency, mutual coupling should be maximized. In several embodiments, variations in coupling with respect to distance and angular misalignment can be simulated using HFSS (Ansys Inc.) simulations, and the point at which coupling decreases by half (−3 dBm power) can be found. Techniques for fabricating an IPG that may be used in accordance with embodiments of the invention include those disclosed in U.S. Patent Publication Number 2022/0158497 entitled “Integrated Energy Harvesting Transceivers and Transmitters with Dual-Antenna Architecture for Miniaturized Implants and Electrochemical Sensors” and U.S. Patent Publication Number 2022/0379124 entitled “Wirelessly Powered Stimulator,” the disclosures of which are hereby incorporated by reference in their entirety, and more particularly the architectures of an IPG are incorporated herein by reference.

### Demodulator

In many embodiments, a demodulator block can be responsible for replicating the timing of the notch, as shown in FIG. 3, in accordance with an embodiment of the invention. The conceptual waveforms of an incident signal 310 and the voltage of the critical nodes 320 in the demodulator are illustrated in FIG. 3. In many embodiments, the circuit can include three source follower replicas. The high end, low end, and transient envelope of the signal are denoted as V_{H}, V_{L}, and V_{ENV}, respectively. The V_{ENV} detection branch may use a relatively small capacitor, C_{SM}, while V_{H} and V_{L} can be extracted on larger capacitors with and without the AC input, respectively. Because of the nonlinearity of the transistors' transfer characteristics, an AC swing applied on a constant gate bias may generate a larger source voltage. The average of V_{H} and V_{L}, V_{M}, can be obtained through a resistive divider, which can thereafter be compared with V_{ENV} to reconstruct the timing of the notch. C_{SM} and C_{LG} can be selected to be 100 fF and 36 pF, respectively. As C_{SM}<<C_{LG}, V_{M} can be considered as constant so that the discharging and charging of C_{SM} determines the delays from the starting and ending points, respectively. A smaller C_{SM} can render a faster transient response yet suffers from a larger noise. In many embodiments, the discharging rate of C_{SM} is independent of the amplitude of the Tx signal as it is determined by the current source generated from a bandgap reference block. The recovered timing signal can then be sharpened by a following buffer 330, as shown in FIG. 3 in accordance with an embodiment of the invention. In certain embodiments, the buffer only causes a sub-ns delay. Although FIG. 3 illustrates a particular circuit architecture of a demodulator, any of a variety of circuit architectures may be utilized as appropriate to the requirements of specific applications in accordance with embodiments of the invention.

### Output Voltage Regulator

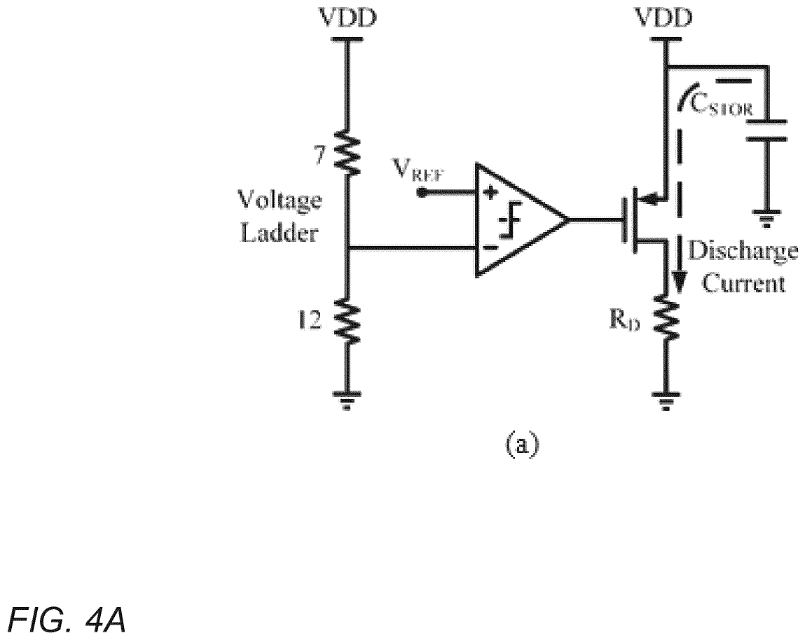

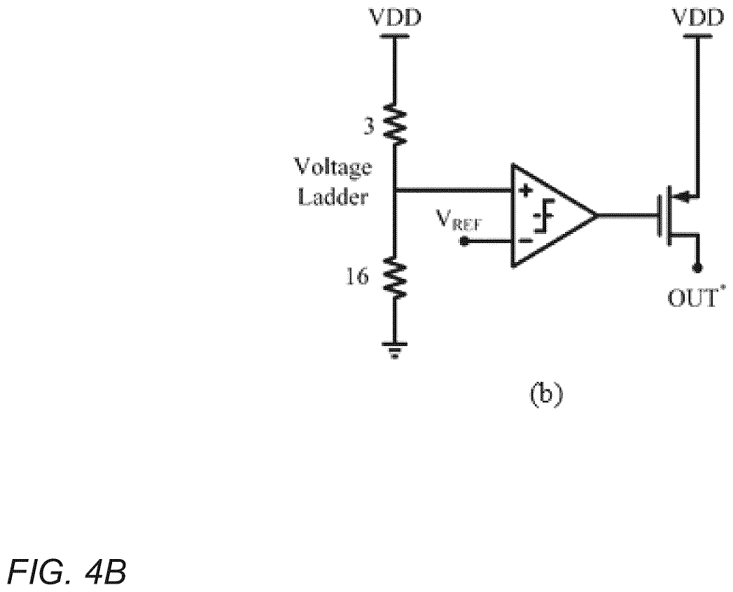

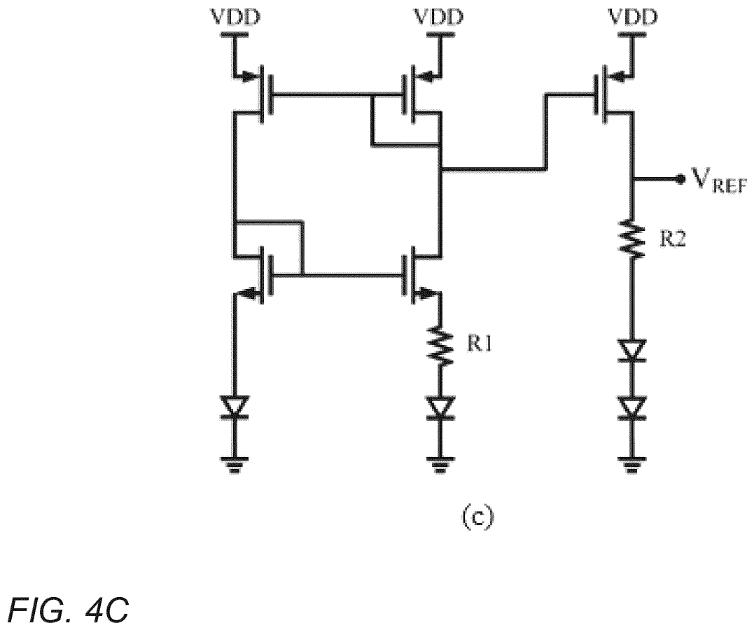

In several embodiments, fractions of VDD can be compared with a constant voltage reference, V_{REF}, so that the amplitude can be regulated within a specific range. Circuits illustrated in FIG. 4A and FIG. 4B in accordance with an embodiment of the invention can be used to determine the high and low bars, respectively. When the supply voltage exceeds 19/12 of V_{REF}, a discharge current path can be enabled through a 65 kΩ resistor, RD, which can rapidly discharge the incident power. On the other hand, in several embodiments, when the amplitude is lower than 19/16 of V_{REF}, OUT* node turns high, which disables the demodulator illustrated in FIG. 3 in accordance with an embodiment of the invention. A bandgap voltage reference circuit in accordance with an embodiment of the invention is shown in FIG. 4C. By tuning R1 and R2, V_{REF} can be designed to be 2.3 V, which can regulate the stimulation amplitude between 2.7 V and 3.6 V. This regulation scheme may eliminate the utilization of LDOs which may turn to be the most static power-consuming block in IMDs. The voltage ladder can be further customized to render a narrower window. In certain embodiments, in the actual operation, an excessive Tx power tends to generate pulses with the maximum amplitude. Although FIG. 4A, FIG. 4B and FIG. 4C each illustrate a particular circuit architecture of an output voltage regulator, any of a variety of circuit architectures may be utilized as appropriate to the requirements of specific applications in accordance with embodiments of the invention.

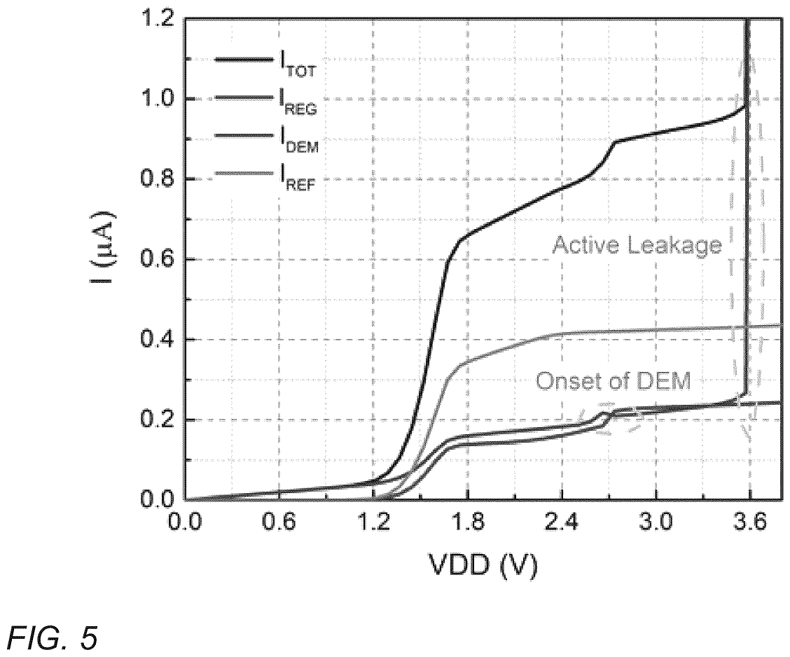

A current consumption of individual blocks is simulated as shown in FIG. 5 in accordance with an embodiment of the invention. With the onset of the demodulator at around 2.7 V, the total current consumption of the IC, I_{TOT}, features a rapid rise (due to the increase of IDEM). When the supply voltage reaches 3.6 V, the leakage path may rapidly discharge the incident power. Below that, the maximum I_{TOT} can be around 950 nA.

### Energy-Harvesting Frontend

In many embodiments, modeling the input impedance of a rectifier as paralleled resistor (R) and capacitor (C) can provide an intuitive insight into the rectifier design for a resonant coupling system. In the subthreshold region, the input impedance of the rectifier may be dominated by the gate capacitances of the MOS transistors. On the contrary, in several embodiments, as the input voltage swing increases, transistors conduct more current so that the input of the rectifier becomes more resistive.

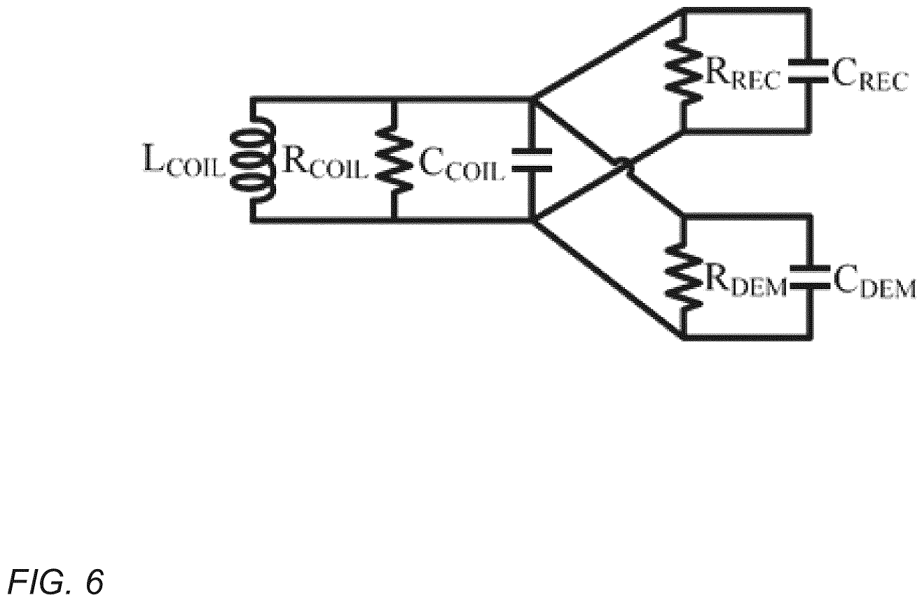

A frontend resonator that includes an Rx coil, rectifier, and demodulator in accordance with an embodiment of the invention is illustrated in FIG. 6. In many embodiments, the Rx coil can be modeled as the parallel configuration of the inductance, L_{COIL}, the loss resistance, R_{COIL}, and the parasitic capacitance, C_{COIL}. In many embodiments, R_{REC} and C_{REC} may represent the input resistance and capacitance of the rectifier, respectively. Similarly, R_{DEM} and C_{DEM} may model the input characteristics of the demodulator. However, in several embodiments, as R_{DEM} and C_{DEM} are simulated to be 1.2 MΩ and 4.7 fF, respectively, they can be omitted. Although FIG. 6 illustrates a particular circuit architecture of an energy-harvesting frontend resonator, any of a variety of circuit architectures may be utilized as appropriate to the requirements of specific applications in accordance with embodiments of the invention.

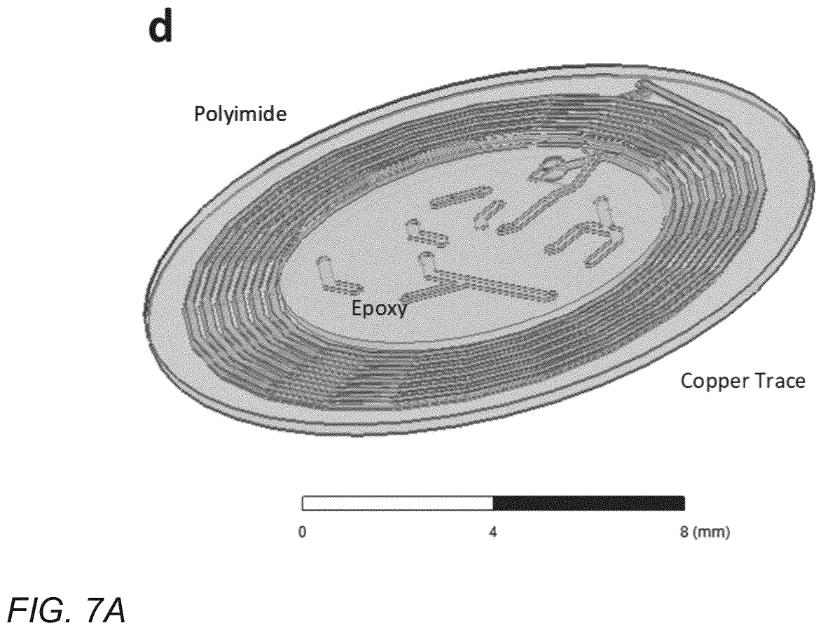

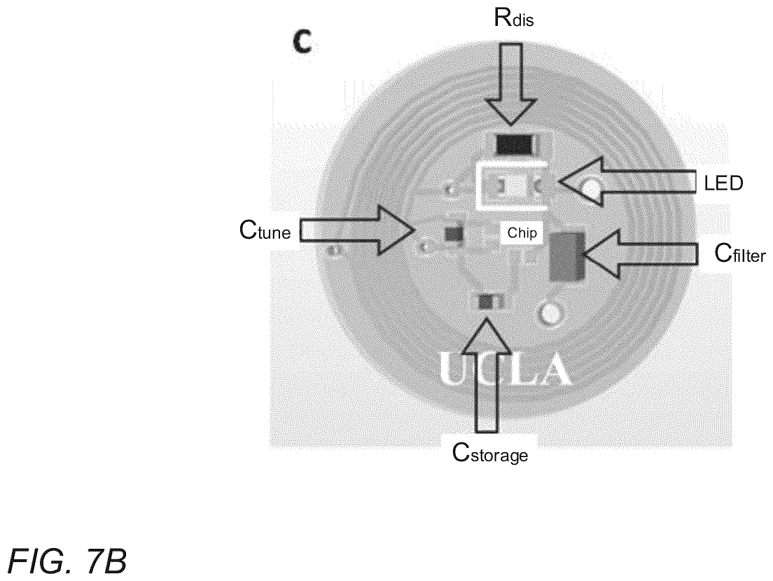

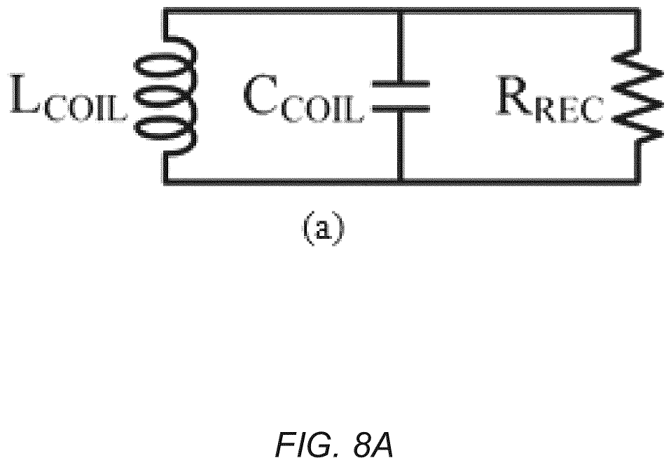

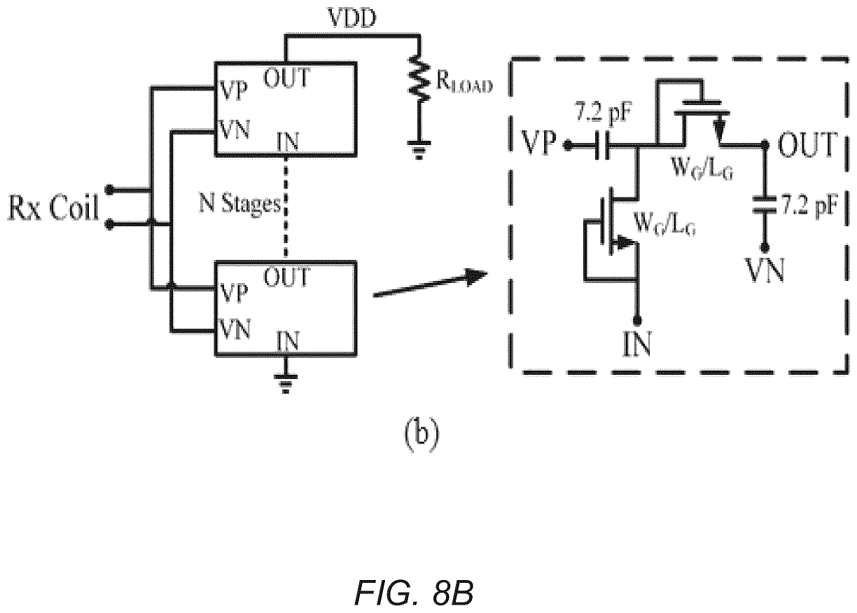

In many embodiments, the Rx coil may dominantly determine the resonant frequency of this resonator. FIG. 7A shows a 3D model and an as-fabricated picture of an Rx coil in accordance with an embodiment of the invention. In certain embodiments, it may reside on 25 μm flexible polyimide substrate with 1 Oz copper traces and feature a twelve-turn design with six turns on the top and bottom layers, respectively. The twelve-turn design enables power harvesting at wavelengths much larger than its dimensions (>1000×). The cuff electrodes (PerenniaFLEX Model 304) and SMD components are assembled on the PCB using silver epoxy (EPO-TEK, H20E). In several embodiments, the size of the Rx coil can be 13 mm in diameter. L_{COIL} can be simulated to be 94.9 nH taking account of all connected traces. As simulations indicate C_{COIL} and R_{COIL} to be an order of magnitude larger than C_{REC} and R_{REC}, respectively, the frontend resonator can be further simplified as illustrated in FIG. 8A in accordance with an embodiment of the invention. The circuit schematic of a Dickson rectifier in accordance with several embodiments is illustrated in FIG. 8B. In certain embodiments, zero-threshold transistors can be used to improve the conversion efficiency. Although FIG. 7 illustrates a particular 3D model of an Rx coil, any of a variety of models may be utilized as appropriate to the requirements of specific applications in accordance with embodiments of the invention. Furthermore, although FIG. 8 illustrates a particular circuit architecture of an energy-harvesting frontend resonator and a Dickson rectifier, any of a variety of circuit architectures may be utilized as appropriate to the requirements of specific applications in accordance with embodiments of the invention.

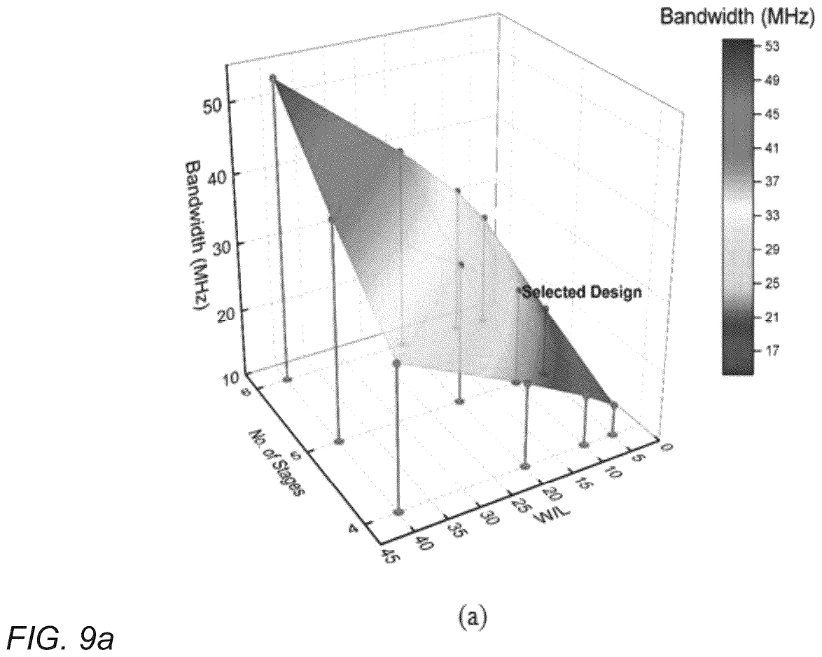

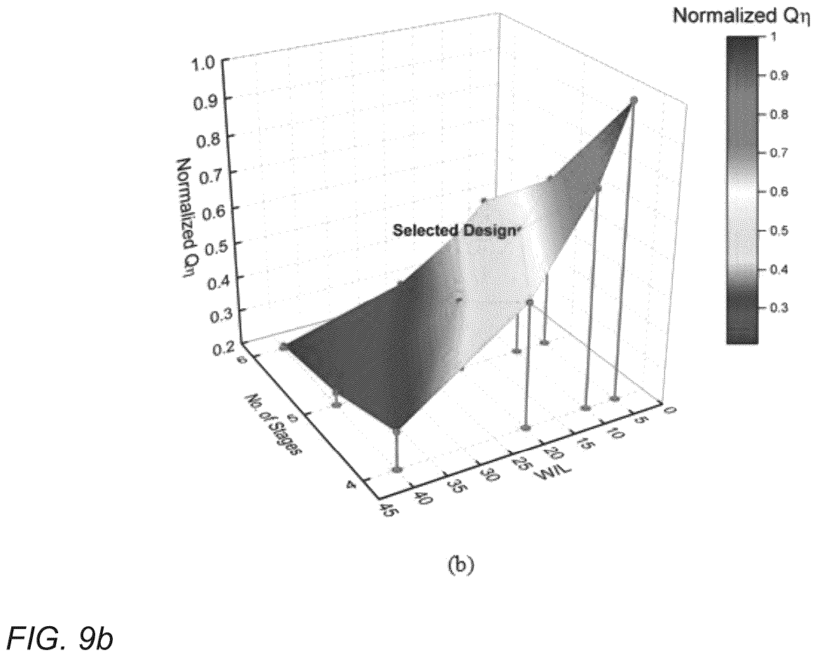

In many embodiments, the design of the rectifier may focus on the tradeoff between the reception sensitivity and bandwidth. Assuming an I_{LOAD} of 5 μA, W_{G}/L_{G} ranging from 2.5 μm/0.5 μm to 20 μm/0.5 μm and the number of stages from 4 to 6 generate different reception bandwidths and sensitivities as shown in FIG. 9 in accordance with an embodiment of the invention. Configurations with more stages and larger W_{G}/L_{G} may render a larger 3 dB-bandwidth of the frontend resonator that can accommodate larger dielectric medium variations, as illustrated in FIG. 9A and FIG. 9B in accordance with an embodiment of the invention. On the contrary, the fewer stages and the smaller W_{G}/L_{G} may lead to a higher reception sensitivity primarily owing to the increased quality factor, Q, as illustrated in FIG. 9B in accordance with an embodiment of the invention. In many embodiments, the reception sensitivity may be compared as the multiplication of Q and the intrinsic conversion efficiency, η, of the rectifier. In many embodiments, a selected design (e.g., W_{G}/L_{G}=5 μm/0.5 μm, N=5) renders a 24 MHz 3 dB-bandwidth and an inherent conversion efficiency of 53% for the rectifier.

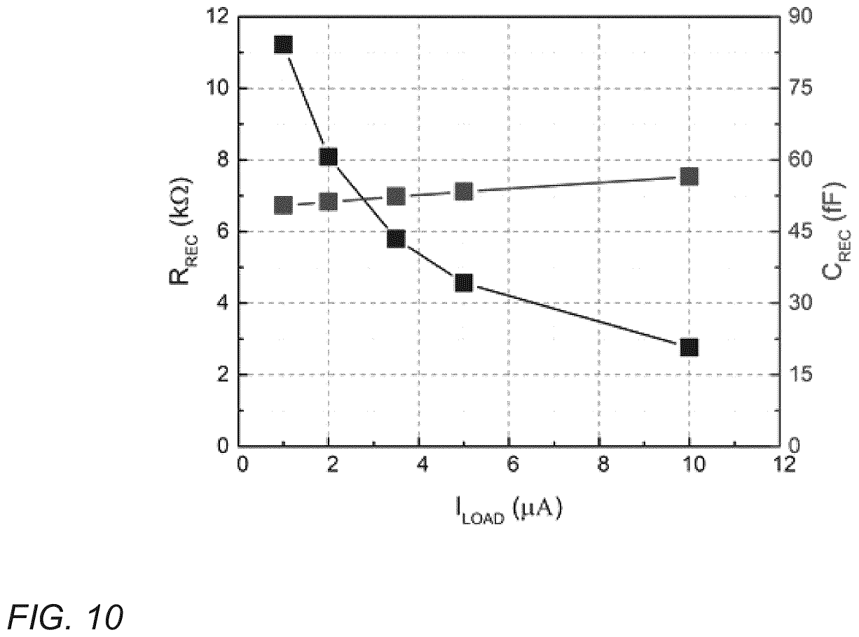

In many embodiments, a selected rectifier design is further simulated to investigate the impacts of I_{LOAD} variations. In certain embodiments, with I_{LOAD} varying from 1 μA to 10 μA, C_{REC} may be remarkably stable at around 50 fF, which verifies the stability of the resonant frequency of the energy-harvesting frontend across a wide range of stimulation loads. On the other hand, R_{REC} may decrease with I_{LOAD}, which indicates an increased reception sensitivity for a lighter load. A simulated dependence of R_{REC} and C_{REC} On I_{LOAD} is demonstrated in FIG. 10 in accordance with an embodiment of the invention.

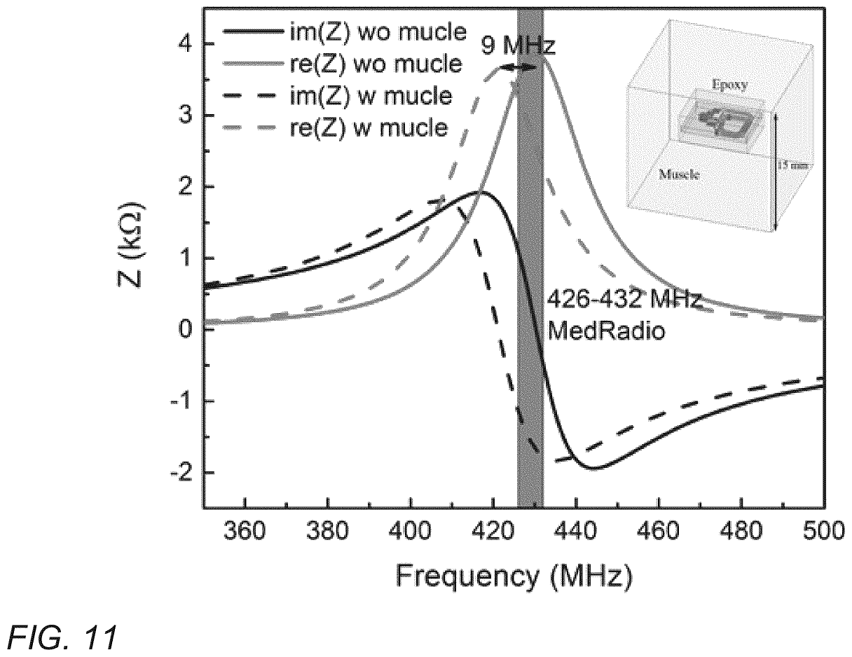

In many embodiments, an IPG assembly can be encapsulated with epoxy. Therefore, the frontend resonator can be simulated within a 3 mm thick epoxy and inside a 1.5 cm muscle cubic to provide an insight into the potential impacts of the dielectric medium variations. In several embodiments, the simulation can be performed with ANSYS and the result shows that the muscle tissue causes a 9 MHz downward drift of the resonant frequency as shown in FIG. 11 in accordance with an embodiment of the invention. In many embodiments, the selected rectifier design succeeds in covering this drift within the 3-dB bandwidth.

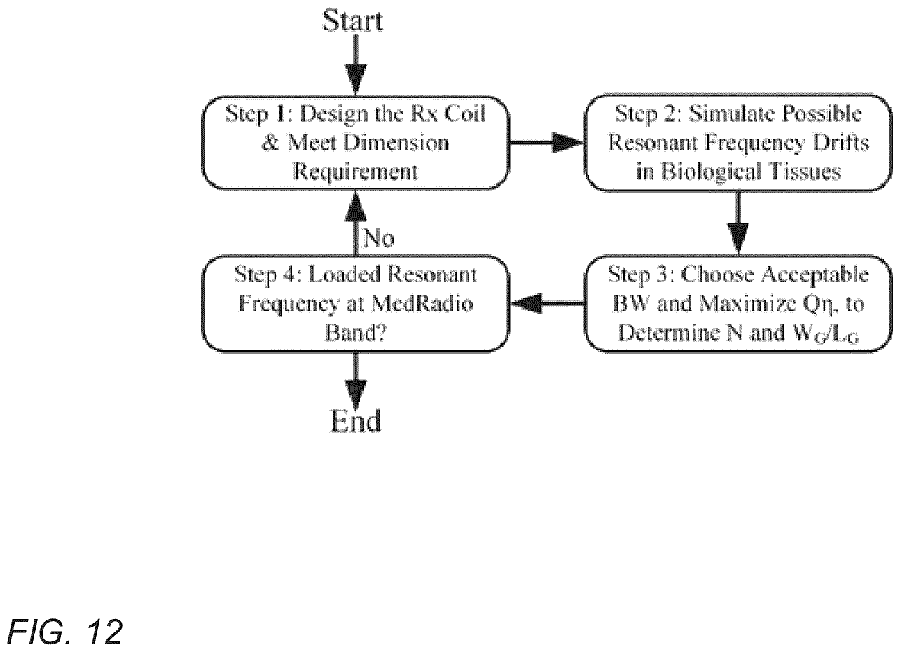

FIG. 12 summarizes a procedure for the co-design of the Rx coil and the rectifier targeting a specific MedRadio band in accordance with an embodiment of the invention. In several embodiments, the Rx coil can play a dominant role in determining the resonant frequency. The rectifier can reach the compromise between the reception sensitivity and bandwidth according to the specific load requirement. In several embodiments, this process may need several iterations of optimization to ensure a certain loaded resonant frequency. Although FIG. 12 illustrates a particular co-design procedure for an Rx coil and rectifier, any of a variety of co-design procedures may be utilized as appropriate to the requirements of specific applications in accordance with embodiments of the invention.

### Electrochemical Impedance Spectroscopy (EIS)

In order to make sure that the electrodes are properly connected to the tissue, the impedance of the tissue can be measured before implantation. Such testing has not traditionally been performed when the stimulator is already implanted in the closed wound, and has generally been performed in open-wound stimulations. Impedance spectroscopy was completed by using a 10 mV AC voltage from 1 Hz to 100 KHz, and the stimulator load impedance was found to be within its operational range.

### Measurement Results

### Fabrication

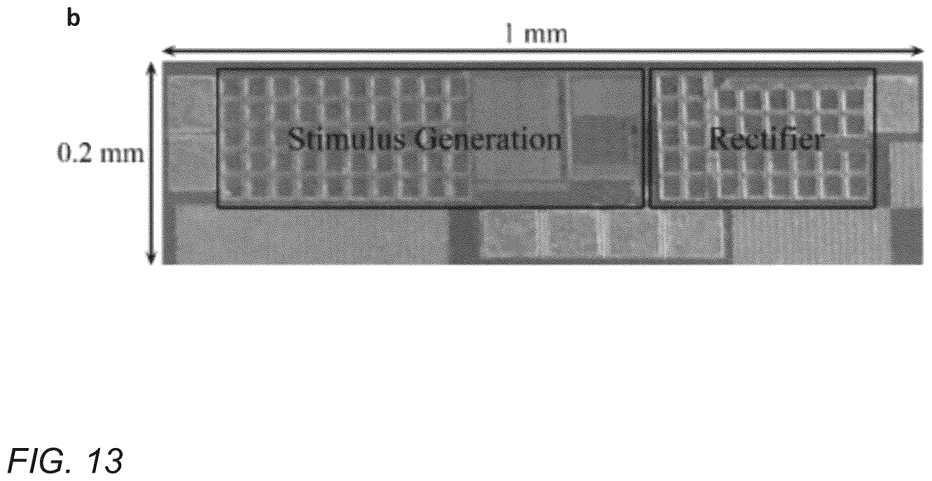

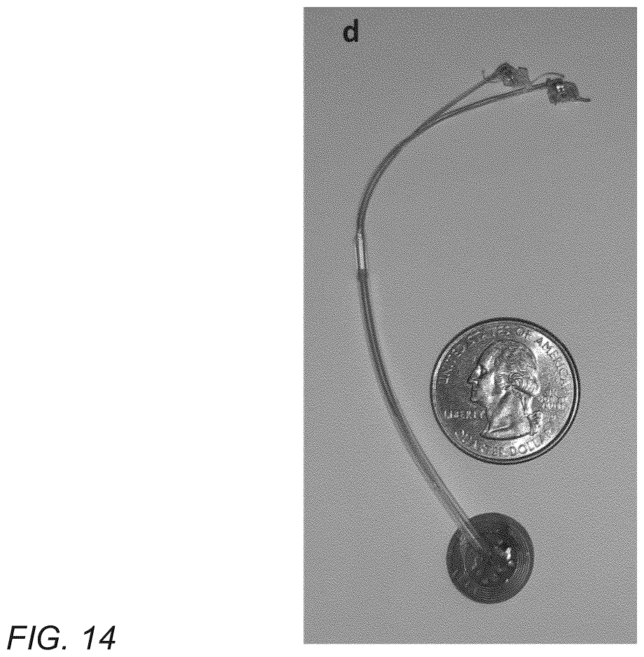

In many embodiments, an IC can be fabricated in TSMC 180 nm CMOS process with a pad-included area of 0.2 mm×1 mm, as shown in FIG. 13 in accordance with an embodiment of the invention. A picture of an IPG assembly in accordance with an embodiment of the invention is shown in FIG. 14. In certain embodiments, epoxy (e.g., EPO-TEK, MED301) can be used to encapsulate the assembly and AWG 22 aluminum plated copper wire of about 5 mm can be utilized as the electrodes for simplicity. Although FIG. 13 illustrates an architecture of an IC, any of a variety of architectures may be utilized as appropriate to the requirements of specific applications in accordance with embodiments of the invention.

### Transmit (Tx) Coil

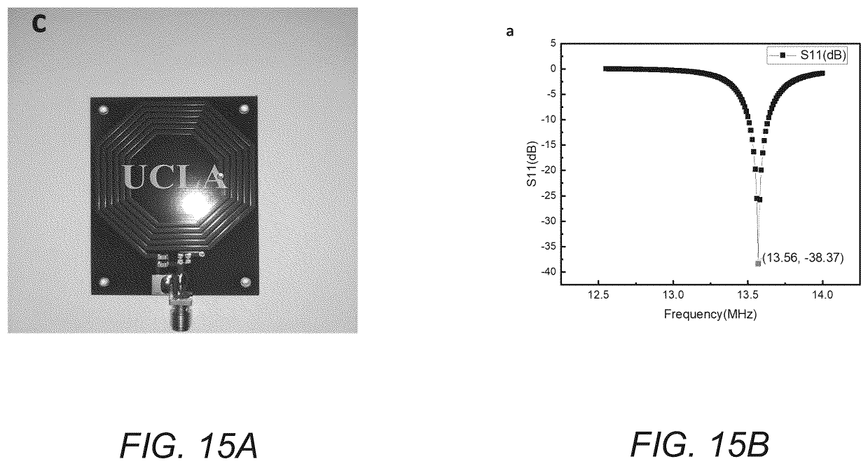

In many embodiments, the Tx coil features a twelve-turn design with six turns on each side, and can be implemented on a 1.6 mm FR4 substrate, as shown in FIG. 15A in accordance with an embodiment of the invention. In several embodiments, the Tx coil has a diameter of approximately 45 mm. In certain embodiments, the wideband Tx coil sweeps at different frequencies to find the resonant frequency of the Rx energy-harvesting frontend to achieve the minimum power to activate the output. The resonant frequency for impedance matching was verified at approximately 13.56 MHz. Although FIG. 15A illustrates a particular circuit architecture of a Tx coil, any of a variety of circuit architectures may be utilized as appropriate to the requirements of specific applications in accordance with embodiments of the invention.

### IPG Output

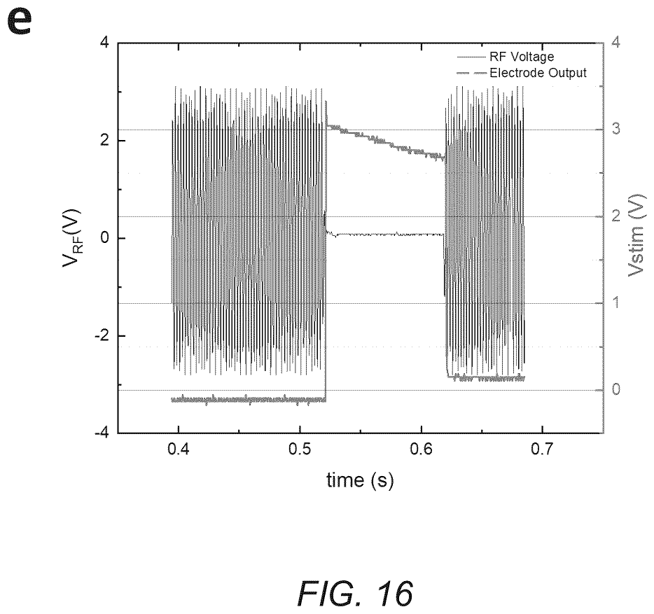

In many embodiments, the electrode impedance can be modeled as a series combination of the tissue/solution resistance, R_{S}, and the double-layer capacitance, C_{DL}, according to works as shown in the inset of FIG. 16 in accordance with an embodiment of the invention. In several embodiments, two electrodes may be immersed in the phosphate buffered solution by approximately 5 mm. RS and CDL can then be characterized to be 1.2 kΩ and 0.6 μF, respectively, with the Stanford Research System SR720 LCR Meter.

In several embodiments, due to the availability of the discrete components, R_{S} of 1.15 kΩ and CDL of 0.6 μF in series may be used as the load of the IPG. In several embodiments, a 6 μs notch may be first applied to the Tx signal, which triggered the output pulse as shown in FIG. 16 in accordance with an embodiment of the invention. In several embodiments, the monophasic waveform has 4.7 μs and 1.4 μs delays compared to the starting and ending points of the notch, respectively. Therefore, the duration of the triggered stimulation can be 3.3 μs shorter than that of the notch. The spike at the onset of the pulse may be an artifact due to parasitic effects of the connection wire.

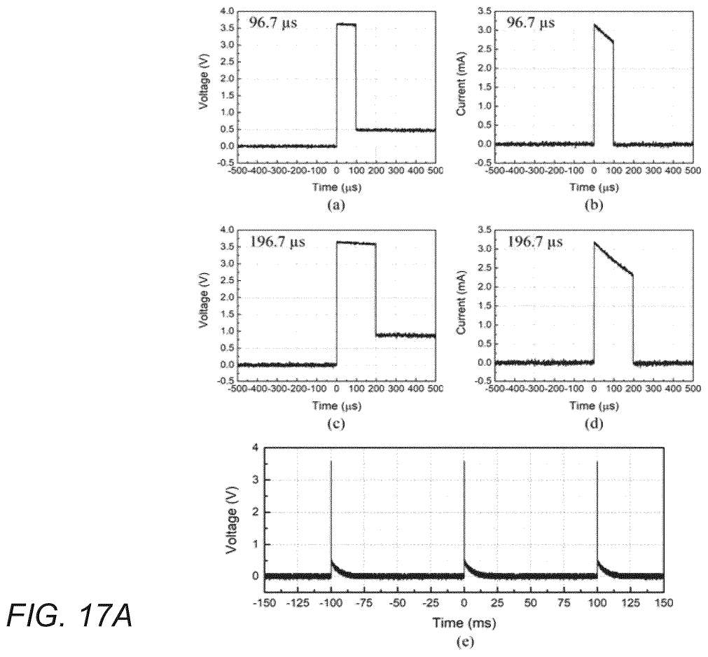

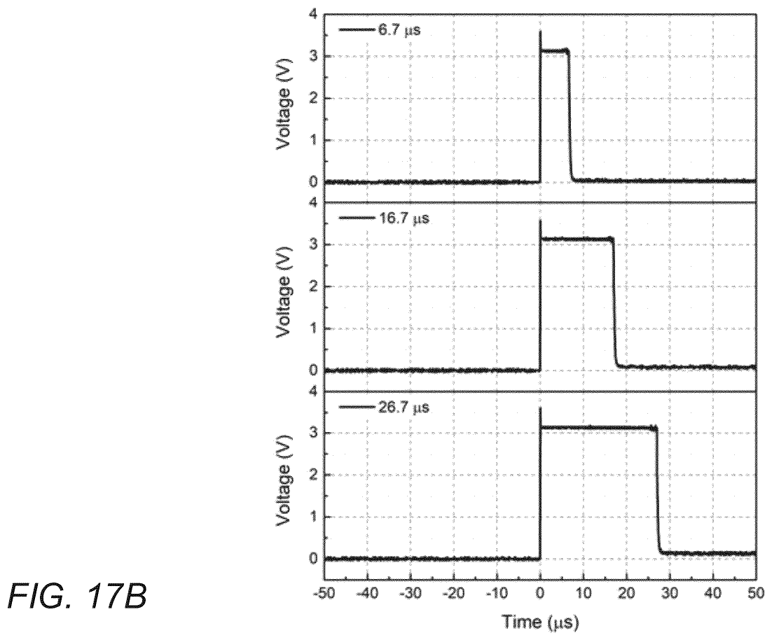

A voltage and corresponding current waveforms for the 96.7 μs and the 196.7 μs pulses are shown in FIG. 17A in accordance with an embodiment of the invention. The injected charge may be temporarily accumulated on CDL so that there appears a post-pulse voltage buildup. In several embodiments, the voltage buildup should not exceed the water delamination window, typically about 1.4 V. In many embodiments, according to this constraint, the pulse width should be kept below 300 μs. The current can be obtained by recording the voltage over the R_{S}, which features an exponentially decaying waveform with the peak of approximately 3.2 mA. In many embodiments, a more comprehensive electrode model may include a charge transfer resistance, R_{CT}, in parallel with C_{DL}, which rapidly discharges the post-pulse potential in saline/tissue. In a typical case, R_{CT} can be around ten times as large as R_{S}. With such R_{CT} of 11 kΩ, the output voltage waveform over multiple cycles is demonstrated at the bottom graph in FIG. 17A in accordance with an embodiment of the invention.

In many embodiments, an LED can be optionally included at the output of the IPG to indicate the occurrence of the output stimulation. In several embodiments, a green LED (e.g., APT1608LZGCK, Kingbright) can be used. In many embodiments, an IPG may be first tested in the air with the Tx power of 1 W. The operating distance can be extended from 50 mm to 100 mm with Tx power at 1 W. The LED may regulate the amplitude of the output pulse at 3.1 V. 6.7 μs, Waveforms of 16.7 μs, and 26.7 μs pulses respectively triggered by 10 μs, 20 μs, and 30 μs notches are demonstrated in FIG. 17B in accordance with an embodiment of the invention.

### Animal Experiment

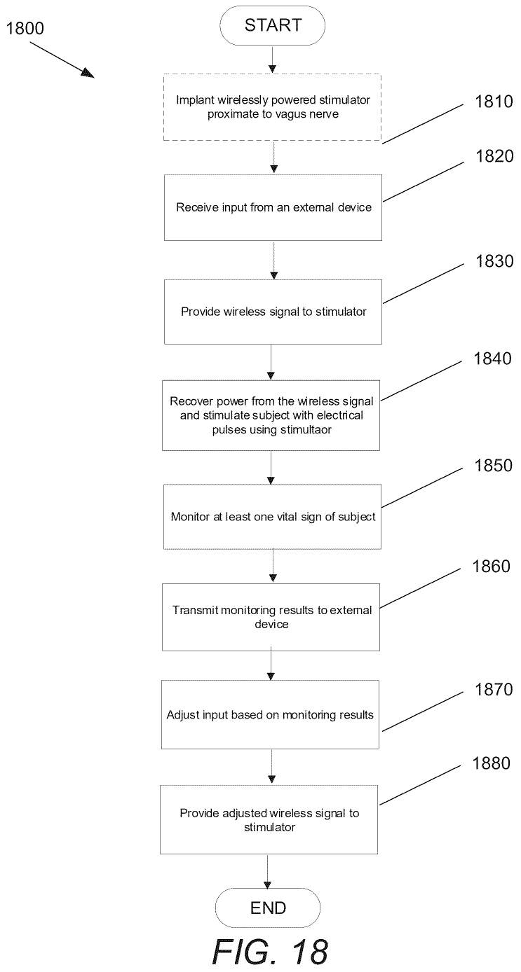

A process for vagus nerve stimulation in accordance with an embodiment of the invention is illustrated in FIG. 18. The results of in vivo studies carried out in three pigs (Sus scrofa, n=3, female, adult 40-44 kg) performed according to a process such as the process illustrated in FIG. 18 will also be discussed here. Process 1800 includes implanting (1810) a Wirelessly Powered Implant (WPI) stimulator into the subject proximate to the vagus nerve. In the study, the WPI devices were implanted on the right side of the neck using standard surgical techniques, and they were powered up using 0.1 W of power at 13.56 MHz. Process 1800 receives (1820) an input from a controller device. The input may include information indicating the desired level of stimulation to be administered to the subject, as discussed further above.

Process 1800 provides (1830) a wireless signal to the WPI. Process 1800 recovers power from the wireless signal and stimulates (1840) the subject with electrical pulses using the WPI. In many embodiments of the invention, the WPI can receive a wireless signal that powers the WPI as well as implies the timing of output electrical pulses to apply, such as enabled by circuits discussed further above. In the study, all pigs were stimulated for a duration of 10 s. The frequency and biphasic pulse width were swept from 3 Hz to 20 Hz and 0.1 ms to 1 ms accordingly. The operation distance could be extended from 50 mm to 100 mm by increasing the power to 1 W. The simulated specific absorption rate (SAR) for 0.1 W of power is 0.77 mW/kg, and it is four orders of magnitude smaller than the 10 W/kg limit specified by IEEE Std C95.1-2005.

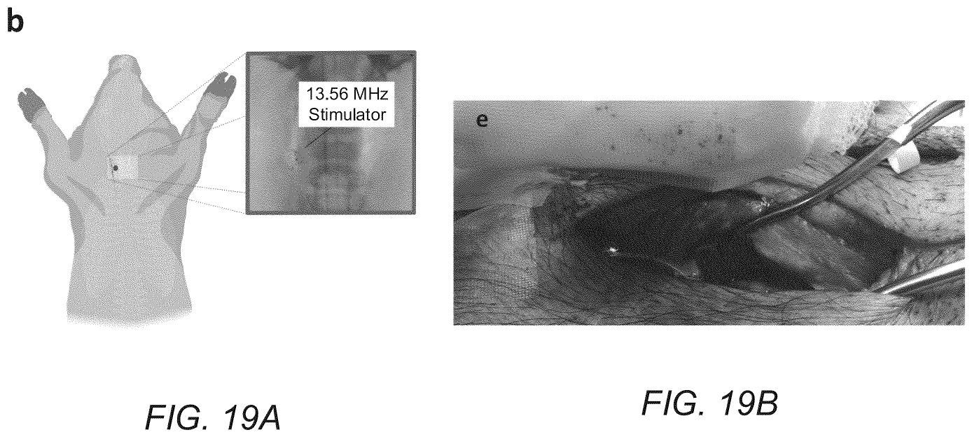

Process 1800 monitors (1850) at least one vital sign of the stimulated subject. In many embodiments, the at least one vital sign includes at least the heart rate (HR) of the subject, which can be calculated, for example, by an ECG measurement as described further above. In the study, vitals of animals were monitored using 3-lead electrocardiogramalse oximetry, arterial blood pressure, end-tidal carbon dioxide, and temperature. The WPI was secured in place with 4-0 Ethilon suture, as shown in the inset of FIG. 19A. The Tx coil delivering the RF signal was placed 3 cm above the hind limb with the source power of 1 W at 13.56 MHz. The connective tissue and skin were sewn covering the WPI. All procedures were in accordance with the National Institute of Health Guide for the Care and Use of Laboratory Animals and were approved by the Animal Research Committee at UCLA.

Process 1800 transmits (1860) monitoring results back to the controller device. In many embodiments, the CCU receives for monitoring results for further analysis. Process 1800 adjusts (1870) input describing the desired stimulation intensity based on the monitoring results. In many embodiments, the adjusting is performed by a model in the CCU. Process 1800 provides (1880) adjusted wireless signal to the stimulator.

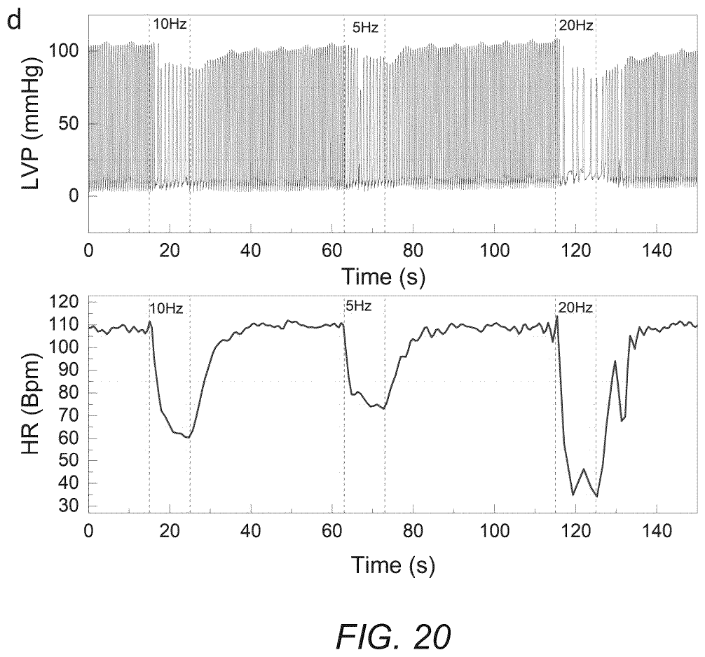

Heart rate (HR) can be calculated by the periodicity of left ventricle pressure (LVP). During the experiments, hemodynamic response was observed for the HR while stimulation was in process. Furthermore, it can be observed that changing the frequency and pulse width of stimulation could produce changes in HR. An example of the HR and LVP response at different frequencies of 5 Hz, 10 Hz, and 20 Hz with a constant pulse width of 0.1 ms is shown in FIG. 20. As expected from FIG. 20, a higher frequency of stimulation induced a stronger response in HR. The maximum change in HR can be calculated by defining HRDelta using Equation (2), and the average changes in HR can also be calculated using the true root mean square (TRMS) of HR by Equation (3):

$${HR}_{Delta} = {HR}_{max} - {Hr}_{min} \qquad ( 2 )$$

$${HR}_{TMRS} = \sqrt{{\int }_{{T}_{start}}^{{T}_{end}} {( HR - {HR}_{Avg} )}^{2} dt} \qquad ( 3 )$$

### Calculation of Charge Delivering

In many embodiments, calculation of the injected amount of charge provides an insight into the proper design of the electrodes for voltage-controlled IPGs. Assuming the voltage buildup on C_{FILTER} to be V_{X} (V_{X} typically much smaller than VDD), the delivered amount of charge with each stimulation equals

$$Δ {Q}_{ch} = ( VDD - {V}_{X} ) ( 1 - {e}^{- {T}_{Pulse} / {R}_{S} {C}_{DL}} ) {C}_{DL} . \qquad ( 1 )$$

Where T_{Pulse} presents the pulse width. The amplitude of the injected current exponentially decays as determined by the time constant according to the electrode model shown in FIG. 16 in accordance with an embodiment of the invention. In the animal experiment, since the pulse amplitude is regulated by the LED at around 3 V, 16.7 μs and 96.7 μs pulses deliver approximately 0.04 μC and 0.23 μC charge, respectively. Multiplying ΔQ_{ch} by the pulse rate, F_{Pulse}, the delivered amount of charge in each second equals

$${Q}_{ch} = {F}_{Pulse} Δ {Q}_{ch} \qquad ( 2 )$$

Note that Q_{ch} is accumulated on C_{FILTER}. Therefore, the passive discharging path should suffice the following relationship,

$${V}_{X} / {R}_{DIS} > {Q}_{ch} \qquad ( 3 )$$

A smaller R_{DIS} in the assembly will ensure a smaller V_{X} that does not evidently hamper the intensity of each stimulation. Many embodiments aim for a μW-level simulation load, R_{DIS} may be selected to be 200 kΩ to ensure a minimum V_{X}. In many embodiments, C_{FILTER} can be 47 μF. A relatively large C_{FILTER} may help to stabilize V_{X}.

### SAR Evaluation

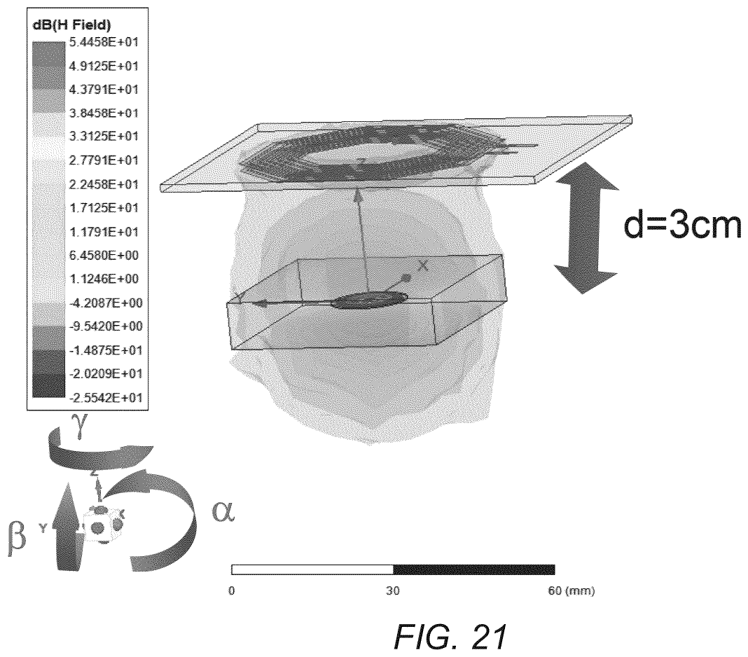

An SAR evaluation may be performed in ANSYS. In many embodiments, placing the Tx coil at a 3 cm distance from the model, the link has −dBm tolerance up to 62° and 46° for α and β misalignment, respectively as shown in FIG. 21 in accordance with an embodiment of the invention. The coil has negligible sensitivity for γ misalignment. In many embodiments, the SAR may be well below the restrictions for localized exposure according to IEEE Std C95.1-2005, i.e., the lower tier of 2 W/kg used for general public and the higher tier of 10 W/kg used for controlled environments, e.g. medical implant use.

### Comparisons

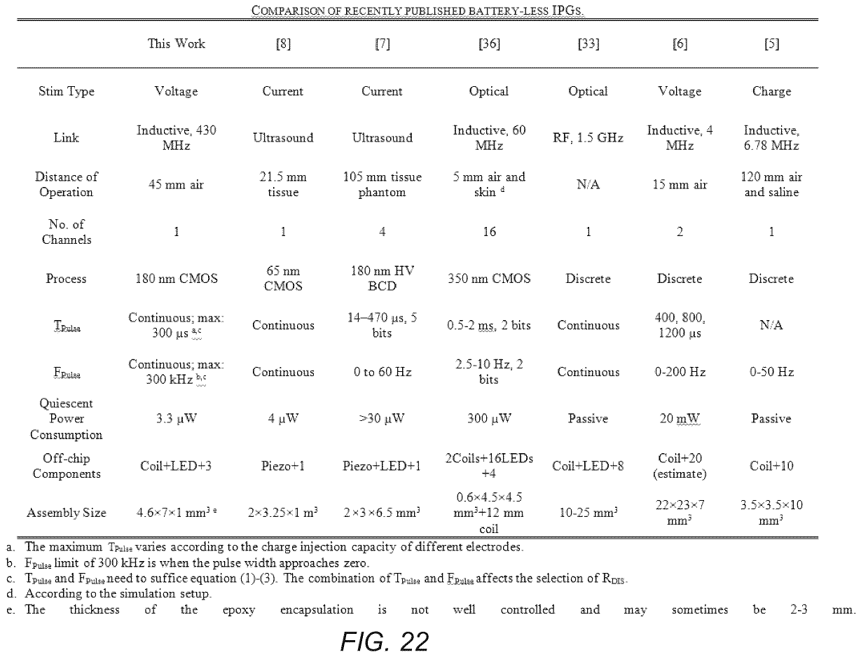

A comparison with recently published miniaturized IPGs is presented in the table illustrated in FIG. 22. Due to the elimination of the coil, ultrasound-based IPGs tend to have smaller form factors. However, their operation typically utilizes ultrasound gel. In addition, there can be concerns with its propagation through air-filled viscera such as the lung and bowel, and obstructions such as bones. Passive circuits have also been investigated to realize energy-efficient IPGs. However, they require sudden bursts of Tx power, which are more prone to violate SAR regulations. To achieve a high reception sensitivity, many embodiments of the IPG consume one of the lowest static powers among active circuitry-based works. The use of MedRadio-band may contribute to the miniaturized form factor of the implant. In many embodiments, replacing the discrete components currently in 0603 SMD packages to 0201 ones can further reduce the overall size by a large portion.

Although specific systems and methods for vagus nerve stimulation with closed-loop monitoring are discussed above with respect to FIGS. 1-22, any of a variety of implementations utilizing the above discussed techniques can be utilized a VNS system with closed-loop monitoring in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. A method for vagus nerve stimulation using at least one wirelessly powered stimulator, the method comprising:
implanting at least one wirelessly powered stimulator proximate to a vagus nerve of a subject, each wirelessly powered stimulator comprising an implantable pulse generator comprising:
a receive antenna;
a rectifier;
an energy storage capacitor;
a demodulator; and
an output voltage regulator;
receiving, at a wearable device, a control input describing stimulation data from a controller device;
providing a radio frequency (RF) signal from the wearable device to the stimulator based on the control input;
receiving and recovering power from the RF signal using the stimulator;
outputting a stimulation that releases energy stored in the energy storage capacitor on a plurality of electrodes in an output pulse having characteristics based on the received RF signal;
generate monitoring results by monitoring, at the wearable device, at least one vital sign of the subject while outputting the stimulation; and
adjusting the outputting the stimulation based on the monitored at least one vital sign.

2. The method of claim 1 wherein adjusting the outputting the stimulation based on the monitored at least vital sign further comprises:
transmitting the monitoring results from the wearable device to the controller device;
adjusting, at the controller device, the control input based on the monitoring results;
adjusting, at the wearable device, the RF signal based on the adjusted control input; and
providing the adjusted RF signal to the stimulator.

3. The method of claim 2, further comprising:
outputting a stimulation that releases the energy stored in the energy storage capacitor on the plurality of electrodes in an output pulse having characteristics based on the received adjusted RF signal.

4. The method of claim 1, wherein adjusting, at the controller device, the control input based on the monitoring results comprises:
determining a target heart rate; and
determining a level of stimulation to maintain the heart rate of the subject at the target heart rate.

5. The method of claim 1, wherein the adjusting of input is performed dynamically based on the monitoring results.

6. The method of claim 5, wherein the dynamic adjustment of input is performed using a data-driven model.

7. The method of claim 1, wherein the at least one vital sign of the subject monitored by the wearable device is the subject's heart rate.

8. The method of claim 1, wherein at least one wirelessly powered stimulator includes a plurality of wireless powered stimulators and the vagus nerve stimulation is performed in more than one location by the plurality of wireless powered stimulations.

9. The method of claim 1, wherein the wirelessly powered stimulator is located within 5 cm of the wearable device.

10. The method of claim 1, wherein the control device comprises an electrocardiogram (ECG) machine.

11. The method of claim 1, wherein the monitoring results comprises an ECG signal of the subject.

12. The method of claim 1, wherein the monitoring results are provided to a coronary care unit (CCU) through the wearable device.

13. The method of claim 1, wherein the wearable device is rechargeable.

14. The method of claim 1, wherein the stimulation can be stopped by a fail-safe mechanism when the monitored vital sign indicates extreme heart rate deviations.

15. The method of claim 1, wherein the characteristics of the output pulse determined by the RF signal are voltage, frequency, and pulse width.

16. A system for vagus nerve stimulation, comprising:
at least one wirelessly powered stimulator, each wirelessly powered stimulator comprising an implantable pulse generator comprising:
a receive antenna;
a rectifier;
an energy storage capacitor;
a demodulator; and
an output voltage regulator;
where each implantable pulse generator is configured to:
receive and recover power from a radio frequency (RF) signal; and
output a stimulation that releases energy stored in the energy storage capacitor on a plurality of electrodes in an output pulse having characteristics based on the received RF signal;
a wearable device configured to:
receive a control input describing stimulation data;
provide a radio frequency (RF) signal from the wearable device to the wirelessly powered stimulator based on the control input; and
generate monitoring results by monitoring at least one vital sign of a subject implanted with at least one wirelessly powered stimulator while outputting the stimulation; and
a controller device configured to:
provide the control input describing stimulation data to the wearable device; and
adjust the control input describing stimulation data based on the monitored at least one vital sign.

17. The system of claim 16, where the wearable device is further configured to:
transmit the monitoring results from the wearable device to the controller device;
adjust the RF signal based on an adjusted control input provided by the controller device; and
provide the adjusted RF signal to the wirelessly powered stimulator.

18. The system of claim 16, where the stimulator is further configured to:
output a stimulation that releases energy stored in the energy storage capacitor on the plurality of electrodes in an output pulse having characteristics based on the received adjusted RF signal.

19. The system of claim 16, where the controller device is further configured to:
determining a target heart rate; and
determines a level of stimulation to maintain the heart rate of the subject at the target heart rate.

20. The system of claim 16, where the controller device is further configured to adjust the control input dynamically based on the monitoring results.

21. The system of claim 16, where the controller device is further configured to adjust the control input dynamically using a data-driven model.

22. The system of claim 16, where the wearable device is further configured to monitor the subject's heart rate.

23. The system of claim 16, where at least one wirelessly powered stimulator comprises a plurality of wireless powered stimulators and the vagus nerve stimulation is performed in more than one location by the plurality of wireless powered stimulations.

24. The system of claim 16, where the wirelessly powered stimulator is located within 5 cm of the wearable device.

25. The system of claim 16, where the control device comprises an electrocardiogram (ECG) machine.

26. The system of claim 16, where the wearable device is further configured to monitor an ECG signal of the subject.

27. The system of claim 16, where the wearable device is further configured to provide the monitoring results to a coronary care unit (CCU).

28. The system of claim 16, where the wearable device is rechargeable.

29. The system of claim 16, further configured to stop the stimulation by a fail-safe mechanism when the monitored vital sign indicates extreme heart rate deviations.

30. The system of claim 16, where the characteristics of the output pulse determined by the RF signal are voltage, frequency, and pulse width.
